(19) United States
(12) Patent Application Publication
(10) Pub. No.: US 2014/0276355 A1
(43) Pub. Date: Sep. 18, 2014

(54) Co-administration of Light and a Therapeutic Agent to Stimulate Dysfunctional Mitochondria Affected By a Neurological Disorder

(71) Applicant: MEDOS INTERNATIONAL SARL, LeLocle (CH)

(72) Inventors: Yanik Tardy, Les Geneveys-sur-Coffrane (CH); Patrik Verstreken, Blanden (BE)

(73) Assignee: MEDOS INTERNATIONAL SARL, LeLocle (CH)

(21) Appl. No.: 14/211,279

(22) Filed: Mar. 14, 2014

Related U.S. Application Data
(60) Provisional application No. 61/783,975, filed on Mar. 14, 2013.

(51) Int. Cl. A61K 31/122; A61N 5/06

(52) U.S. Cl. CPC A61K 31/122; A61N 5/0613
USPC 604/20

(57) ABSTRACT

A method and device for stimulating a dysfunctional mitochondria in a human brain afflicted with a neurological disorder by stimulating the dysfunctional mitochondria at more than one level of its electron transport chain by co-administering more than one type of external stimulation such as the co-administration of both light and a therapeutic agent such as vitamin K2.

### CROSS-REFERENCE TO RELATED APPLICATIONS

This application claims the benefit of U.S. Provisional Application No. 61/783,975, filed on Mar. 14, 2013, which is herein incorporated by reference in its entirety.

### BACKGROUND OF THE INVENTION

1. Field of the Invention

The present invention relates to mitochondria. More particularly, the invention relates to the stimulation of dysfunctional metabolism of mitochondria affected by a neurological disorder by the co-administration of light (e.g., photobiomodulation/low level light therapy) and a therapeutic agent a compound (such as a drug or biological compound) used for the treatment of a disease or for improving the well-being of an organism).

2. Description of Related Art

Mitochondria are found in virtually all eukaryotic cells and function to generate cellular energy in the form of adenosine triphosphate (ATP) by oxidative phosphorylation. These mitochondria are responsible for providing most of the required ATP for cells. ATP is the chemical responsible for energy release within cells that drives a multitude of cellular and physiological functions. Structurally, mitochondria are composed of a double lipid bilayer with a phospholipid outer membrane 2 and an inner membrane 1 which surrounds the intracompartmental matrix 4, as depicted in FIG. 4. The cristae 3 (space between the two membranes) is important in, and contains the major units of oxidative phosphorylation. An electron transport chain (ETC) is composed of four complexes (e.g., Complex I, II, III, IV) located in the inner mitochondria membrane. The function of the chain is to generate cellular energy in the form of ATP. This is accomplished by the transport of electrons between complexes causing proton (H+ ions) movement from the matrix 4 to the intermembrane space 3 generating a proton concentration (electrochemical) gradient used by ATP-synthase to produce ATP, as is illustrated in FIG. 5.

Parkinson's disease affects millions of individuals worldwide and is increasing with the aging population. This progressive, neurodegenerative condition has been linked with mitochondria dysfunction and inhibition of the ETC, as discussed in the publication by Keane et al., “Mitochondrial Dysfunction in Parkinson's Disease,” SAGE-Hindawi Access to Research, Parkinson's Disease, Vol. 2011, Article ID 716871, 18 pages, which is herein incorporated by reference in its entirety. Due to the central role of the mitochondria in energy production, mitochondria dysfunction results in a depletion of cellular energy levels. Studies have detected in Parkinson's patients mitochondria dysfunction accompanied by Complex I deficiency and impaired electron transfer (e.g., an increase in the release of electrons from the ETC into the mitochondria matrix).

Much research is targeted today in developing a pharmacological agent in the treatment of Parkinson's disease. However, a significant hurdle exists in the development a pharmacological agent for the treatment of neurodegenerative disorders in that any drug delivered to the brain, such as a drug targeting the mitochondria, must be able to cross the blood brain barrier (BBB) in order to reach the central nervous system (CNS). A physical barrier (i.e., the BBB) inside the brain protects the CNS from harmful substances in the blood such as viruses, parasites, chemicals and biological substances. Thus, any developed pharmacological agent has to not only address the neurodegenerative disorder but also be able to cross the BBB.

It is desirable to develop a method for the treatment of Parkinson's and other neurological disorders having a dysfunctional affect on the mitochondria metabolism by external stimulation of the mitochondria metabolism at more than one level in the respiratory chain (or ETC).

### SUMMARY OF THE INVENTION

An aspect of the present invention is directed to a method for stimulating the metabolism of a dysfunctional mitochondria in it human brain afflicted with a neurological disorder by stimulating, the dysfunctional mitochondria at more than one level of the electron transport chain by co-administering more than one type of external stimulation. The levels of the electron transport chain correspond to one of Complex I, Complex II, Complex III, Complex IV.

Still another aspect of the present invention is directed to a method for stimulating a dysfunctional mitochondria in a human brain afflicted with a neurological disorder by stimulating dysfunctional mitochondria at more than one level of the electron transport chain by co-administering more than one type of external stimulation. The first level of the electron transport chain that is stimulated corresponds to Complex I and is externally stimulated by delivery of a therapeutic agent (e.g., Vitamin K2) to the brain. The second level of the electron transport chain externally stimulated by exposure of the mitochondria to a controlled amount of light (e.g., photobiomodulation) is Complex IV.

### BRIEF DESCRIPTION OF THE DRAWING

The foregoing and other features of the present invention will be more readily apparent from the following detailed description and drawings of illustrative embodiments of the invention wherein like reference numbers refer to similar elements throughout the several views and in which:

FIG. 1A is a partial cross-sectional view of a first embodiment of an exemplary implantable delivery system for the co-administration of a controlled amount of light and a therapeutic agent targeting different levels in the respiratory chain of the mitochondria in accordance with the present invention;

FIG. 1B is an end view of the exemplary implantable delivery system in FIG. 1A;

FIG. 2A is a partial cross-sectional view of a second embodiment of an exemplary implantable delivery system for the co-administration of a controlled amount of light and a therapeutic agent targeting different levels in the respiratory chain of the mitochondria in accordance with the present invention;

FIG. 2B is an end view of the implantable delivery system in FIG. 2A;

FIG. 3 is a schematic of an exemplary delivery system in accordance with the present invention for the co-administration of a controlled amount of light and a therapeutic agent targeting different levels of the respiratory chain of the mitochondria;

FIG. 4 is a prior art schematic of the mitochondria structure;

FIG. 5 is a prior art schematic of the electron transport chain in the mitochondria;

FIG. 6 is an exemplary transdermal deliver system in accordance with the present invention for the co-administration of a controlled amount of light and a therapeutic agent targeting different levels of the respiratory chain of the mitochondria; and

FIG. 7A is graphical test data conducted on dysfunctional mitochondria from a. Drosophila model illustrating that, when compared to non treated controls, the greatest restoration of locomotor function (as measured by flight capacity) results from the synergistic effect of exposure to both Vitamin K2 and light;

FIG. 7B is graphical test data conducted on dysfunctional mitochondria from a Drosophila model illustrating that, when compared to non-treated controls, the greatest increase in ATP levels results from the synergistic effect of exposure to both Vitamin K2 and light.

### DETAILED DESCRIPTION OF THE INVENTION

In accordance with the present invention, dysfunctional mitochondria whose energy production (metabolism) has been reduced due to a neurodegenerative disorder (such as, but not limited to. Parkinson's disease) is improved by subjecting the mitochondria to two different types of external stimuli affecting different levels of the respiratory chain (ETC). The first external stimulus is exposure of the mitochondria in the brain to a controlled amount of light (e.g., photobiomodulation (PBM)) {also known as low level light therapy (LLLT)} in order to: (i) increase photo-activity of the ETC thereby increasing the proton gradient across the mitochondria membrane; (ii) increase energy expenditure; and (iii) increase production of ATP. While the second external stimulus is delivery of a therapeutic agent (e.g., a drug and/or biological compound (e.g., Vitamin K2)) to the mitochondria in the brain. Each external stimulus is discussed in detail separately below. It is advantageous that the present invention method stimulates mitochondria function at different levels or components (e.g., Complex I, II, III, IV) of the ETC.

In particular, the light selected for PBM or LLLT is at a predetermined range of wavelengths that modulates the activity of the ETC, and therefore, increases the proton transfer towards the intermembrane space of the mitochondria. Preferably, PBM or LLLT uses monochromatic light (typically red to near infra-red (NIR) light at a wavelength of approximately 600 nm to approximately 1100 nm) at relatively low intensity (i.e., having no ablative or thermal affect). Different light sources such as, but not limited to, one or more laser diodes, light emitting diodes (LEDs) or other emitters may be employed to produce the controlled low level of light (LLL) used in PBM.

A publication by Huang et al. entitled “Biphasic Dose Response In Low Level Light Therapy,” International Dose Response Society (2009), Vol. 7, pp. 358-383, which is herein incorporated by reference in its entirety, describes the affects LLLT has on the mitochondria of cells. As disclosed in the publication, the mechanism of PBM by red to NIR light at the cellular level has been ascribed to the activation of specific mitochondria respiratory chain components. In particular, a strong body of experimental evidence suggests that Complex IV (cytochrome C oxidase (COX)) in the ETC is the primary photoacceptor of light in the red to NIR spectral range. Irradiation of the mitochondria to PBM therefore photostimulates Complex IV (COX) in the ETC resulting in increased absorption of photons that, in turn, accelerates electron transfer reactions and increases ATP production. It has been discovered that Nitric Oxide (NO) in cells can bind to Complex IV (COX) and hinder respiration. When the mitochondria of cells are irradiated with LLL, the accepted Photon displaces/releases (photodisassociates) nitric oxide bound to Complex IV, allowing oxygen binding (i.e., increased oxidative binding) to Complex IV, increased activation of the ETC and increased production of ATP. A number of downstream transcription and regulation processes are then activated (as discussed in the Huang et al. publication) and result in increased cell viability (as discussed in the publication by Shaw et al. entitled “Neuroprotection of Midbrain Dopaminergic Cells in MPTP-Treated Mice after Near-Infrared Light Treatment,” The Journal of Comparative Neurology (2010), Vol. 518, pp. 25-40), which is herein incorporated by reference in its entirety).

A recent publication by Vos, M. et al., entitled “Vitamin K2 Is a Mitochondrial Electron Carrier That Rescues Pink 1 Deficiency,” Science, (Stine 2012), pp. 1306-1310, which is herein incorporated by reference in its entirety, discloses the results of studies using a Drosophila (fruit flies) model for Parkinson's disease. The studies demonstrate that mitochondria dysfunction associated with Parkinson's disease can be rescued by the administration of Vitamin K2. Human UBIAD1 localizes to mitochondria and converts vitamin K1 to vitamin K2. Drosophila UBIAD1 (Heixuedian) has been identified as a modifier of pink1, a gene mutated in Parkinson's disease that affects mitochondria function. Vitamin K2 is a membrane-bound electron carrier that is necessary and sufficient to transfer electrons in Drosophila mitochondria. Heixudian mutants showed severe mitochondria defects that were rescued by Vitamin K2 by transferring electrons in Drosophila mitochondria, resulting in more efficient ATP production. Specifically, Vitamin K2 acts as an electron carrier facilitating electron transport downstream of a eukaryotic ETC complex (e.g., Complex I (NADH dehydrogenase), resulting in improved mitochondria oxygen consumption, energy production, and production of ATP. Furthermore, Vitamin K2 is a particularly advantageous biological compound in targeting mitochondria in the brain due to its ability to easily cross the BBB.

The co-administration of the two external stimuli (e.g., a controlled amount of light and a therapeutic agent such as Vitamin K2) in accordance with the present invention creates a synergistic long term affect on the metabolism of the mitochondria by acting at different levels of the respiratory chain. As used herein the term “co-administration” is broadly defined as each external stimulus being delivered in time continuously or discontinuously, wherein the delivery in time of the multiple stimuli relative to one another occurs simultaneously (completely overlapping), intermittently/alternatingly (no overlapping), partially overlapping or any combination thereof. Accordingly, the method and timing of the delivery of each external stimulus may therefore be independently controlled.

FIG. 3 is an exemplary schematic diagram of the implantable delivery system 300 in accordance with the present invention for the co-administration of a controlled amount of light and a therapeutic agent to targeted mitochondria. System 300 includes a co-administering delivery device 320 for delivery of the external stimuli to the targeted mitochondria. Device 320 is controlled by control unit 310 either via a wired or wireless electrical interface. Control unit 310 is powered by a power source 330 (e.g., battery). The parameters associated with co-administering delivery device 320 are selected or adjusted using the electronic control circuit 350 of the control unit 310. A therapeutic agent (e.g., Vitamin K2) to be administered by the delivery device 320 may be replenished from a reservoir 340 associated with the external control unit 310. One or more light sources 360 (e.g., one or more laser diodes or LEDs) generate the LLL. It is also contemplated and within the intended scope of the present invention for the reservoir and/or light source to be separate from, rather than integrated into, the external control unit 310. Control unit 310 may be either fully implantable or connected through a transcutaneous port when treatment is required.

A first embodiment of the present inventive integrated implantable co-administering delivery device for stimulation of the mitochondria metabolism is illustrated in FIGS. 1A & 1B. The delivery device includes a light guide (e.g., optical fiber system) including an outer cladding 10 with an inner hollow core 30 disposed concentrically therein defining therebetween a core-cladding, boundary 20. Concentrically disposed within the inner hollow core 30 is a catheter 40 having a lumen 50 defined longitudinally therethrough for delivery of the therapeutic agent (e.g., Vitamin K2) to the targeted mitochondria, for example, in the brain. The same targeted mitochondria cells are also stimulated by being irradiated with LLL generated by the light source 360 and transmitted through the light guide. In an alternative configuration of the embodiment depicted in FIG. 1A, catheter 40 may be eliminated altogether whereby the therapeutic agent is delivered via the inner hollow core 30 of the optical fiber itself.

In operation, the implantable delivery system is introduced into the body proximate a target site, preferably in the brain. Once positioned at the targeted location, the light source 360 is energized thereby irradiating the targeted site in the brain to the LLL. The therapeutic agent, for example, vitamin K2, is dispensed from the reservoir 340 and delivered via the lumen 50 to the same targeted site.

A second embodiment of an exemplary implantable delivery device for co-administering a controlled amount of light and a therapeutic agent for stimulation of the mitochondria metabolism is illustrated in FIGS. 2A & 2B. This device differs from that of the first embodiment in that the LLL is delivered on the inside of the device, while the therapeutic agent is delivered, on the outside of the device. The delivery device includes a catheter 240 having a lumen 250 defined longitudinally therethrough. Disposed concentrically or simply free floating within the lumen 250 is a light guide (e.g., optical fiber) 210. The LLL generated by the light source 360 is directed or focused using mirrors and other optical devices into the entrance of the light guide 210. Simultaneously, a therapeutic agent (e.g., Vitamin K2) is delivered through the lumen 250 in a region around the optical fiber.

In still another alternative embodiment, the optical fiber and catheter may be integrated into a single delivery device arranged side-by-side.

The present invention is not limited to only implantable delivery systems. Instead, any known method of delivery for each of the two external stimuli is contemplated and within the intended scope of the present invention for targeting more than one level of the respiratory chain of the targeted mitochondria cells. Instead of being implanted, the method of delivery for one or both stimuli may be transdermal. For example, the therapeutic agent may be delivered via a transdermal patch 610 (as shown in FIG. 6), or otherwise delivered orally or via injection. Similarly, rather than be implanted, the controlled amount of light may be delivered using a non-invasive external light delivery device 630. Referring to FIG. 6, device 630 includes a power source 650 (e.g., battery) and one or more light sources 610, preferably one or more lasers, light emitting diodes (LEDs). Light source 610 produces light within at a specific wavelength or within a predetermined range of wavelengths that maximizes activation of the different levels in the respiratory chain of the mitochondria. However, any conventional light source producing light in the specific wavelength to maximize activation of the desired complexes in the respiratory chain of the mitochondria may be utilized. The number, size, arrangement and placement of the one or more light sources may vary, as desired, depending on the size and location of the targeted mitochondria cells. Rather than a fixed light source, it is also contemplated to use a variable light source whose wavelength may be varied, as desired. Preferably, the light generated by light source 110 is in the red or NIR wavelength range since these wavelengths correspond to the excitation frequency of the Cytochrome C Oxydase (complex IV of the ETC). Advantageously, soft tissue (e.g., skin, fat muscle) is relatively transparent to light within these specified wavelength ranges. Light within the red or infrared frequency wavelength range generally has a penetration depth of approximately 1 cm-approximately 2 cm in soft tissue. The device 630 has one or more windows or diffusers 140 in its housing through which the light produced by the source 610 is able to pass either completely unobstructed or only partially obstructed so that at least some light is able to pass therethrough. The diffusers homogenize the light intensity over the illuminated area. A single diffuser 640 in FIG. 6 spans the entire width of the device 630; however, the size, shape, number and arrangement of the diffusers 640 may be modified, as desired.

A controller 620 such as a CPU, microprocessor or processor is also preferably included in the device 630 for varying, as desired, one or more control parameters associated with the light produced by the light source 610. By way of illustrative example, the one or more control parameters adjusted by the controller 620 may include at least one of intensity, wavelength, duration, pulse vs. continuous, etc. The parameters associated with each of the LEDs may be controlled either independently or altogether as a group, by controller 620. All circuitry and components including controller 620 may be disposed within a single housing, as shown in FIG. 6. Alternatively, some of the circuitry and/or components may be disposed within a device separate from that of the light source. In such latter case, the two separate devices may communicate via a conventional wired or wireless communication interface.

In use device 630 is positioned with one or more transparent diffusers 640 proximate the skin of the patient oriented so as to bathe in the generated light the targeted mitochondria cells. Light produced by the source 610 passes through the one or more diffusers 640 of the housing 630 as well as the soft tissue and bathing the targeted mitochondria cells. Power source 650 (e.g. battery) is used to power the controller 620, the one or more light sources 610 and all other electronic circuitry.

Different methods of delivery or a common method of delivery for each of the external stimuli is contemplated with the scope of the present invention. Although only two external stimuli are disclosed, as being co-administered by the present inventive delivery system and method, more than two external stimuli may be employed to stimulate more than one level of the respiratory chain of the same targeted mitochondria cells.

To demonstrate the synergistic effect of exposure to both a potential therapeutic agent (Vitamin K2 in this data) and exposure to light (808 nm continuous laser diode during 100 s with an irradiance of 25 mW/cm2 in this data) a Drosophila model for Parkinson disease has been used. Flight assays were conducted using male flies in batches of 5 flies each. Flies were placed in an empty vial (5 cm×10 cm), gently tapped and scored visually for their ability to fly. Flies able to fly were given a score of I while those that did not fly were given a score of 0. FIG. 7A shows less than 15% of the non-treated controls have the ability to fly due to their dysfunctional mitochondria. Systemic administration of Vitamin K2 or light treatment administrated separately restored flight capability for about 30% of the animals. The combination of both treatments (e.g., Vitamin K2 and light) provided more than 50% restoration of the locomotor impairment. Similar synergistic effect was observed with ATP levels (FIG. 7B).

Thus, while there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps that perform substantially the same function, in substantially the same way, to achieve the same results be within the scope of the invention. Substitutions of elements from one described embodiment to another are also fully intended and contemplated. It is also to be understood that the drawings are not necessarily drawn to scale, but that they are merely conceptual in nature. It is the intention, therefore, to be limited only as indicated by the scope of the claims appended hereto.

Every issued patent, pending patent application, publication, journal article, book or any other reference cited herein is each incorporated by reference in their entirety.

What is claimed is:

1. A method for stimulating a dysfunctional mitochondria metabolism in a human brain afflicted With a neurological disorder, said method comprising the step of:
stimulating the dysfunctional mitochondria at more than one level of an electron transport chain by co-administering more than one type of external stimulation.

2. The method in accordance with claim 1, wherein the levels of the electronic transport chain correspond to one of Complex I, Complex II, Complex III, Complex IV.

3. The method in accordance with claim 2, wherein stimulation of the dysfunctional mitochondria occurs at two levels of the electronic transport chain.

4. The method in accordance with claim 3, wherein one of the two levels of the electronic transport chain that is stimulated is Complex I.

5. The method in accordance with claim 4, wherein the external stimulation is the delivery of a therapeutic agent to the brain.

6. The method in accordance with claim 5, wherein the therapeutic agent is Vitamin K2.

7. The method in accordance with claim 3, wherein one of the two levels of the electronic transport chain that is stimulated is Complex IV to increase production of adenosine triphosphate.

8. The method in accordance with claim 1, wherein the external stimulation is exposure of the brain to light stimulation.

9. The method in accordance with claim 3, wherein the two levels of the electronic transport chain that are stimulated is Complex I and Complex IV.

10. The method in accordance with claim 1, wherein the stimulating step comprises co-administering light stimulation and delivery of a therapeutic agent to the brain to stimulate the mitochondria metabolism.

11. The method in accordance with claim 10, wherein the therapeutic agent is Vitamin K2.

12. The method in accordance with claim 1, wherein each of the more than one type of external stimulation is delivered continuously or discontinuously.

13. The method in accordance with claim 1, wherein the more than one type of external stimulation is delivered in time relative to one another simultaneously, intermittently or partially overlapping.

14. The method in accordance with claim 13, wherein the more than one type of external stimulation is delivered either via an invasive delivery system or a non-invasive delivery system.

15. A system for stimulating a dysfunctional mitochondria metabolism in a human brain afflicted with a neurological disorder, said system comprising:
a delivery system configured to stimulate the dysfunctional mitochondria at more than one level of an electron transport chain by co-administering more than one type of external stimulation.

16. The system in accordance with claim 15, wherein the delivery system includes a light delivery device configured to transmit light or stimulation of the dysfunctional mitochondria.

17. The system in accordance with claim 15, wherein the delivery system includes a therapeutic agent delivery device configured to deliver a therapeutic agent for stimulation of the dysfunctional mitochondria.

18. The system in accordance with claim 16, wherein the delivery system includes a therapeutic agent delivery device configured to deliver a therapeutic agent for stimulation of the dysfunctional mitochondria.

19. The system in accordance with claim 18, wherein the light delivery device is configured to deliver a therapeutic agent for stimulation of the dysfunctional mitochondria.

20. The system in accordance with claim 15, wherein the more than one type of external stimulation is light and vitamin K2.
